(19) United States
(12) Patent Application Publication
(10) Pub. No.: US 2012/0029570 A1
(43) Pub. Date: Feb. 2, 2012

(54) FACET ARTHROPLASTY DEVICE

(75) Inventor: Alain TORNIER, Saint-Ismier (FR)

(73) Assignee: CLARIANCE, Dainville (FR)

(21) Appl. No.: 13/193,975

(22) Filed: Jul. 29, 2011

Related U.S. Application Data
(60) Provisional application No. 61/382,651, filed on Sep. 14, 2010.

(30) Foreign Application Priority Data
Jul. 29, 2010 (FR) 10/03179

(51) Int. Cl. A61B 17/70

(52) U.S. Cl. 606/265

(57) ABSTRACT

The facet arthroplasty device according to the present invention comprises linking rods (2) for linking together connectors (3, 6) that are held respectively in the vertebral bodies of each treated vertebra V by way of anchoring screws (4), while each connector (3) has means for immobilizing and guiding a sleeve (5) that allows said connector (3) freedoms of movement in rotation R, R1 and/or in lateral tilting B about a vertical axis xx' and/or vv' of the anchoring screw (4), which has been fixed beforehand in the corresponding vertebral body.

The present invention relates to a facet arthroplasty device and more particularly to a device for mounting and immobilizing an anchoring screw in a connector.

The object of the facet arthroplasty device according to the present invention is to improve the way in which an anchoring screw is mounted in a connector, while at the same time ensuring that the latter is able to pivot angularly about said anchoring screw when the latter is anchored in the osseous body of a vertebra.

The facet arthroplasty device according to the present invention comprises linking rods for linking together connectors that are held respectively in the vertebral bodies of each treated vertebra V by way of anchoring screws, each connector having means for immobilizing and guiding a sleeve that allows said connector freedoms of movement in rotation R, R1 and/or in lateral tilting B about a vertical axis xx' and/or vv' of the anchoring screw, which has been fixed beforehand in the corresponding vertebral body.

The facet arthroplasty device according to the present invention comprises a sleeve, which has a spherical outer profile and a continuous inner bore of conical profile.

The facet arthroplasty device according to the present invention has a linking rod, which comprises a cylindrical part that is continued by an inclined part having in cross section a square or rectangular profile.

The facet arthroplasty device according to the present invention shows that each connector is formed by a body comprising a first bore, which allows the cylindrical part of the linking rod to be guided in translation, and a second bore, which is formed in a continuation for receiving the sleeve and the anchoring screw.

The facet arthroplasty device according to the present invention shows that the second bore has two internal vertical indents arranged opposite each other and opening into a seat formed on the inner periphery of the second bore.

The facet arthroplasty device according to the present invention shows that the indents and the seat formed inside the second bore of each connector have a spherical profile complementing the outer profile of the sleeve, in such a way that said sleeve can move freely inside the second bore in a rotation movement R and/or in a lateral tilting movement B with respect to the vertical axis xx' of said bore and/or in a rotation movement R1 with respect to the vertical axis vv' of the anchoring screw.

The facet arthroplasty device according to the present invention comprises a sleeve, which has a height greater than that of the continuation of the connector.

The facet arthroplasty device according to the present invention shows that each anchoring screw has a convex profile between a first anchoring part and a second anchoring part, which convex profile is continued, in the direction of the second anchoring part, by a conical profile designed to cooperate with the conical bore of the sleeve in order to constitute a Morse taper connection permitting the immobilization of said sleeve on said anchoring screw.

The facet arthroplasty device according to the present invention shows that the inclined part of the linking rod is held, by way of a locking screw, inside connectors that have been fixed beforehand in the superjacent vertebrae V2 of the spinal segment to be treated.

The facet arthroplasty device according to the present invention has, between the connectors, a transverse linking device composed of connectors, which are fixed on the cylindrical part of each linking rod by way of locking screws, and of a transverse rod arranged perpendicularly with respect to said linking rod and allowing said connectors to be linked together.

In the following description, which will give a better understanding of the invention, of the features of the invention and of the advantages that the invention is likely to afford, reference is made to the attached drawings, which are given as non-limiting examples and in which:

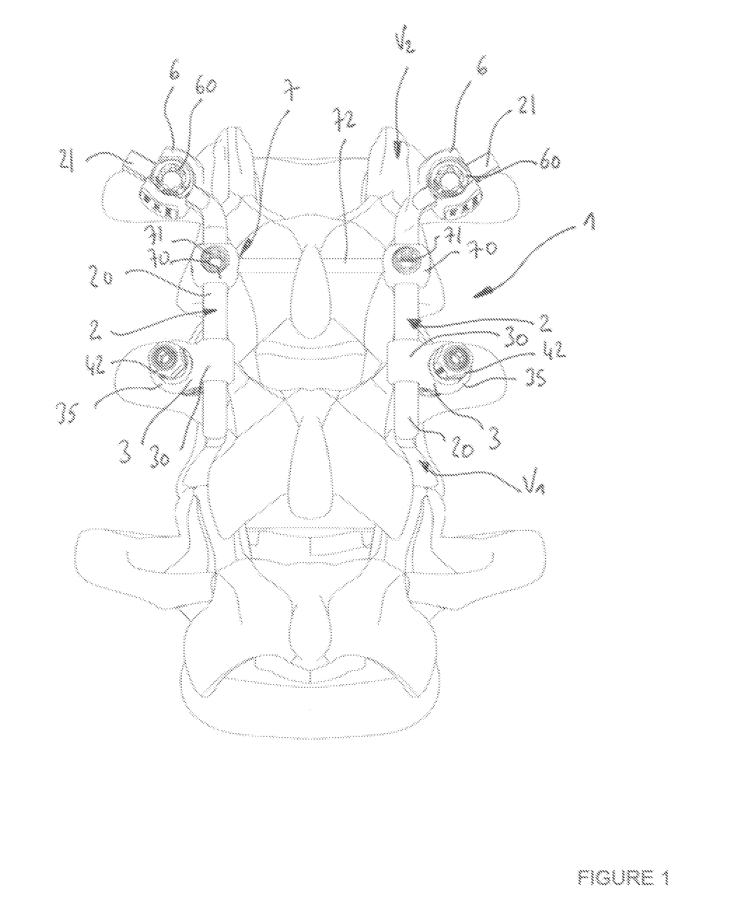

FIG. 1 is a perspective view showing the facet arthroplasty device according to the present invention, arranged on those vertebrae of a spinal column that are to be treated.

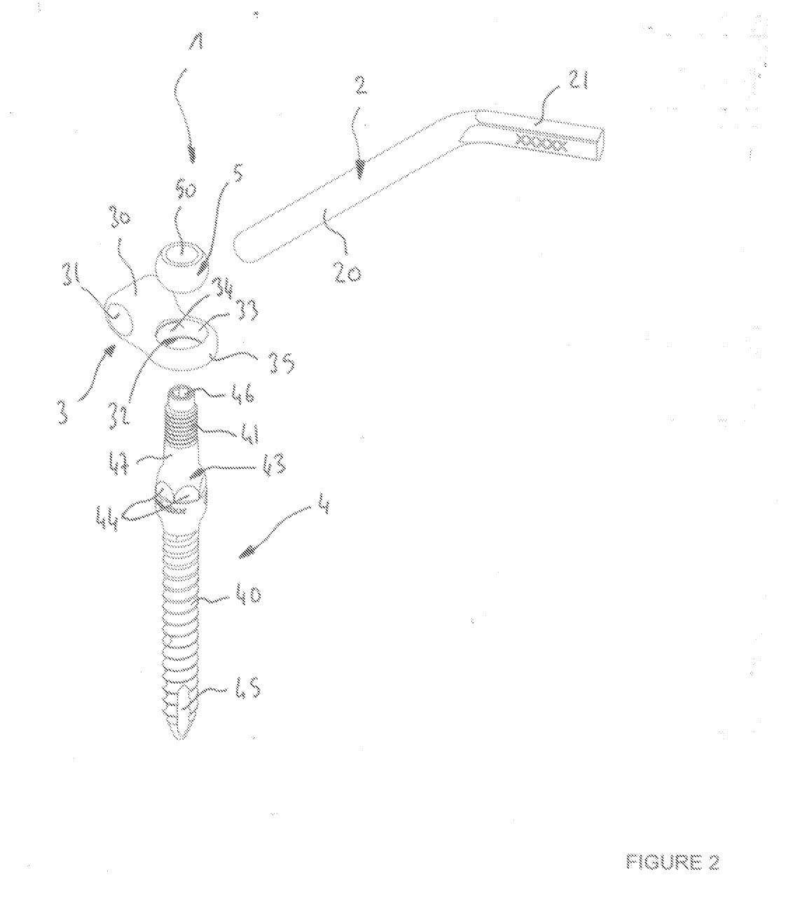

FIG. 2 is an exploded perspective view illustrating the elements constituting the facet arthroplasty device according to the present invention.

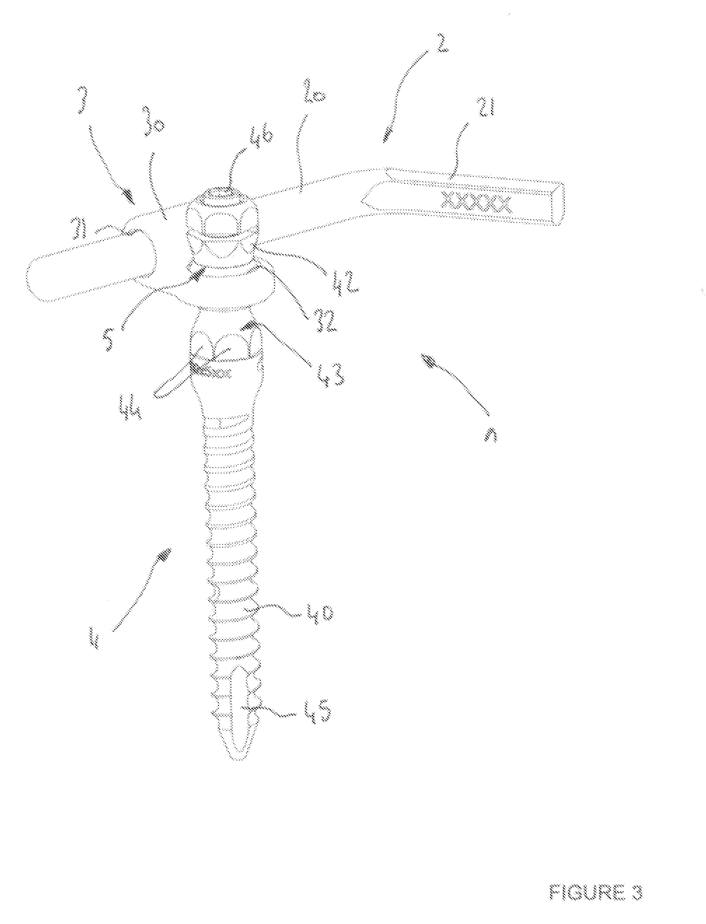

FIG. 3 is a perspective view showing the assembled elements of the facet arthroplasty device according to the present invention.

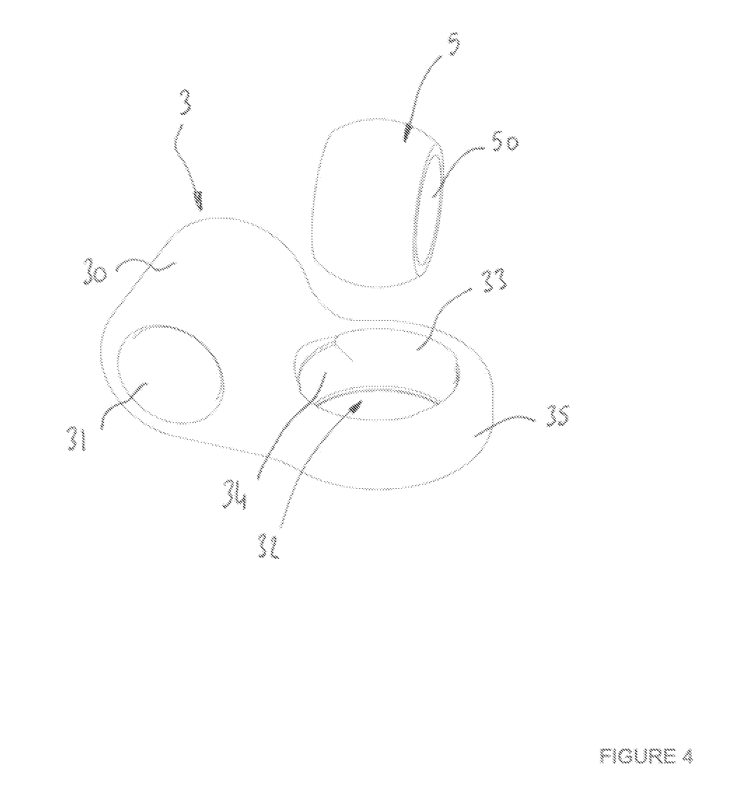

FIGS. 4 to 7 are views showing a sleeve of spherical profile being fitted inside a connector of the facet arthroplasty device according to the present invention.

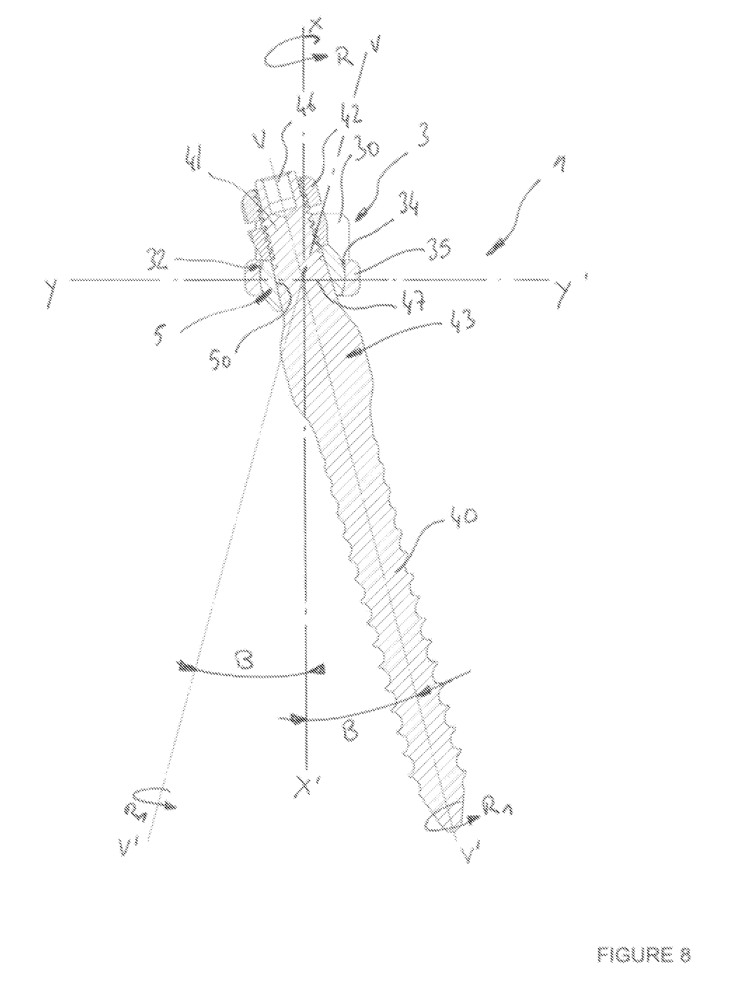

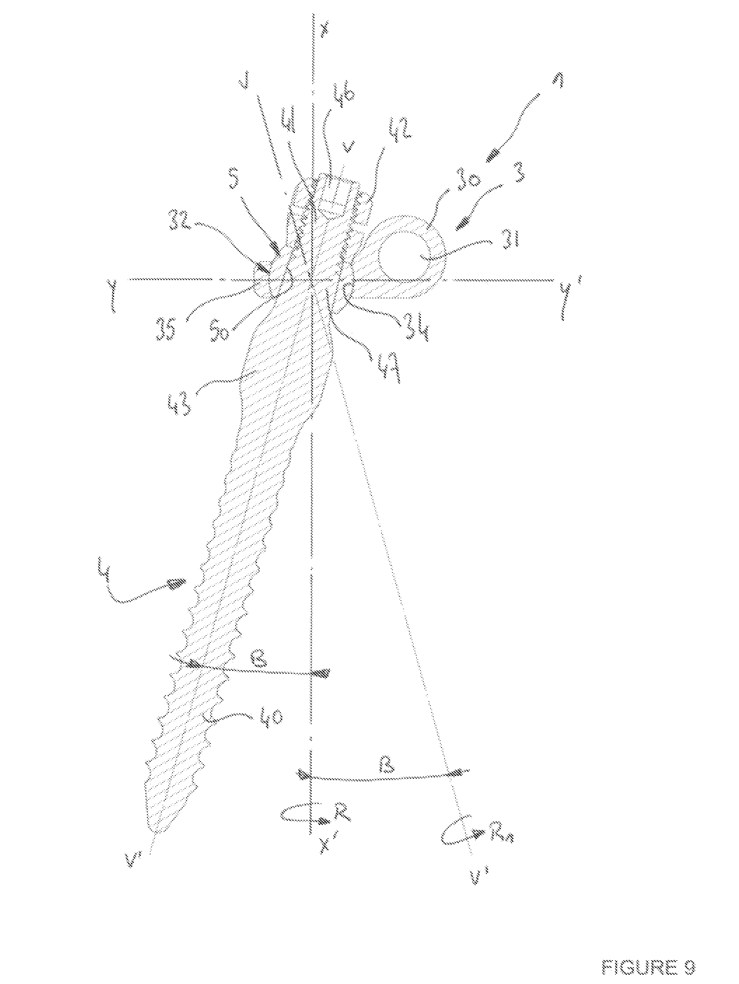

FIGS. 8 and 9 are sectional views illustrating the anchoring screw being fitted in the connector of the facet arthroplasty device according to the present invention.

A facet arthroplasty device 1 is shown in FIGS. 1 to 3, comprising linking rods 2 for linking together connectors 3, 6 that are held respectively in the vertebral bodies of each treated vertebra V1, V2 by way of anchoring screws 4.

The linking rod 2 of the facet arthroplasty device 1 comprises a cylindrical part 20, which is continued by an inclined part 21 having in cross section a square or rectangular profile on which a locking screw 60 bears in order to block it in translation and rotation inside the corresponding connectors 6 that have been fixed beforehand in the superjacent vertebrae V2 of the spinal segment to be treated.

The facet arthroplasty device 1 can be linked between the connectors 3, 6 by a transverse linking device 7 composed of connectors 70, which are fixed on the cylindrical part 20 of each linking rod 2 by way of locking screws 71, and of a transverse rod, 72, which is arranged perpendicularly with respect to said linking rod 2 and allows said connectors 70 to be linked together.

The cylindrical part 20 of the linking rod 2 is also designed to cooperate freely with the connectors 3, that is to say it is able to move freely in translation inside each connector depending on the angular movements of the superjacent vertebra V1 and of the subjacent vertebra V2 with respect to each other.

Each connector 3 is composed of a body 30 comprising a first bore 31, for freely guiding the cylindrical part 20 of the linking rod 2, and of a second bore 32, which is formed in a continuation 35, in such a way that said second bore 32 is arranged in a direction perpendicular to that of said first bore.

Each anchoring screw 4, allowing the connectors 3 to be fixed in the subjacent vertebra V2 of the spinal segment to be treated, is composed of a first anchoring part 40, which has a self-tapping thread and is continued vertically by a second anchoring part 41 with a mechanical thread for receiving a tightening nut 42.

Between the first anchoring part 40 and the second anchoring part 41, the anchoring screw 4 has a convex profile 43 comprising, on its perimeter, sectors 44 that define a zone of engagement for a tool for driving said screw in rotation in order to fix it in the vertebral body of the corresponding subjacent vertebra V2.

Above the convex profile 43, the anchoring screw 4 has a connection zone 47 to the second anchoring part 41, which has the mechanical thread and is of conical profile.

At the free end of the first anchoring part 40, the anchoring screw 4 has vertical notches 45 interrupting the self-tapping thread and ensuring unforced tapping of the bone.

At the free end of the second anchoring part 41 and in the continuation of the mechanical thread, the anchoring screw 4 has an inner seat 46 of hexagonal profile for receiving a tool that blocks said anchoring screw in rotation when the nut 42 is tightened.

FIGS. 4 to 7 show a connector 3 of the facet arthroplasty device 1 according to the present invention, which connector 3 comprises, inside the second bore 32, immobilizing and guiding means for receiving a sleeve 5 by which said connector is allowed with respect to the anchoring screw 4, or vice versa, freedoms of movement in rotation and in tilting as a function of the movements of the superjacent and subjacent vertebrae V1, V2.

For this purpose, the immobilizing and guiding means, starting from the upper edge of the continuation 35 of the connector 3, are formed by two vertical indents 33 arranged opposite each other and opening into a seat 34 formed on the inner periphery of the second bore 32.

The second bore 32 formed in this way is designed to receive the sleeve 5, which has a spherical outer profile and has a continuous inner bore 50 of conical profile.

The indents 33 and the seat 34 formed inside the second bore 32 of the connector 3 have a spherical profile complementing the outer profile of the sleeve 5.

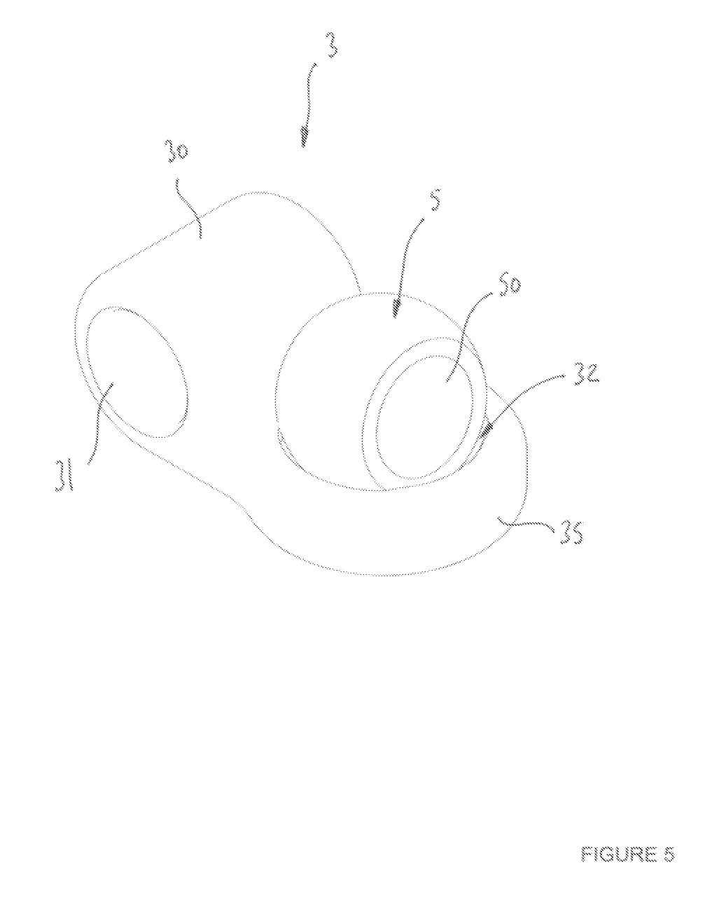

For this purpose, the sleeve 5 is introduced inside the second bore 32 in a vertical direction, in such a way that its outer profile cooperates with the indents 33 until abutment inside the seat 34 (FIG. 5).

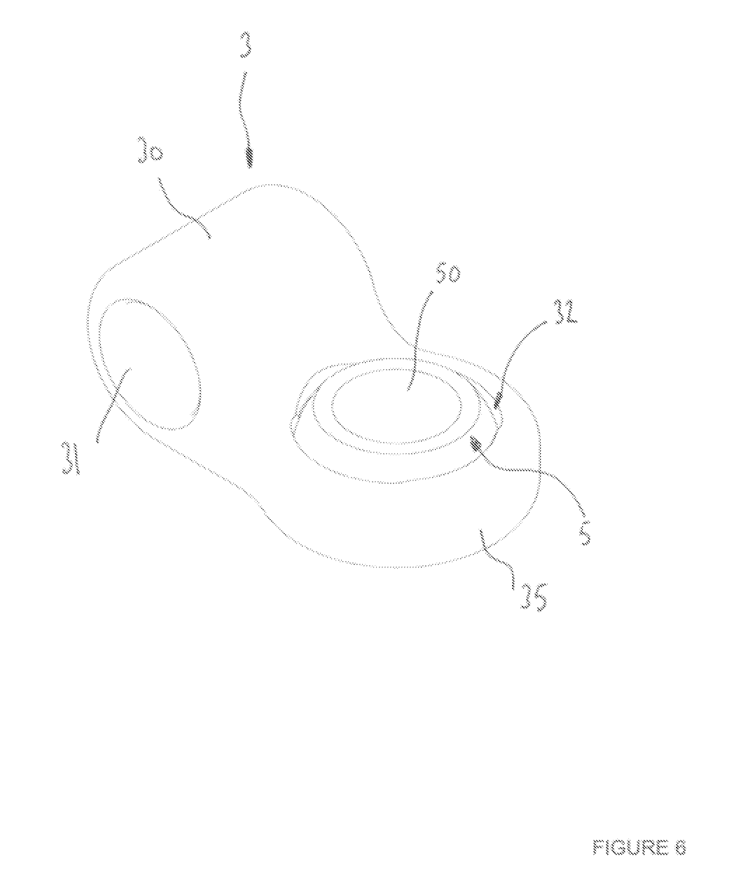

The sleeve 5 is then tilted inside the second bore 32, in such a way that its outer profile cooperates only with the complementary profile of the seat 34 (FIG. 6).

After it has been tilted, the sleeve 5 is positioned inside the second bore 32 in a horizontal position parallel to the horizontal plane containing the continuation 35 of the connector 3, in such a way that the more open base of the conical profile of the inner bore 50 is directed away from the upper edge of said continuation 35.

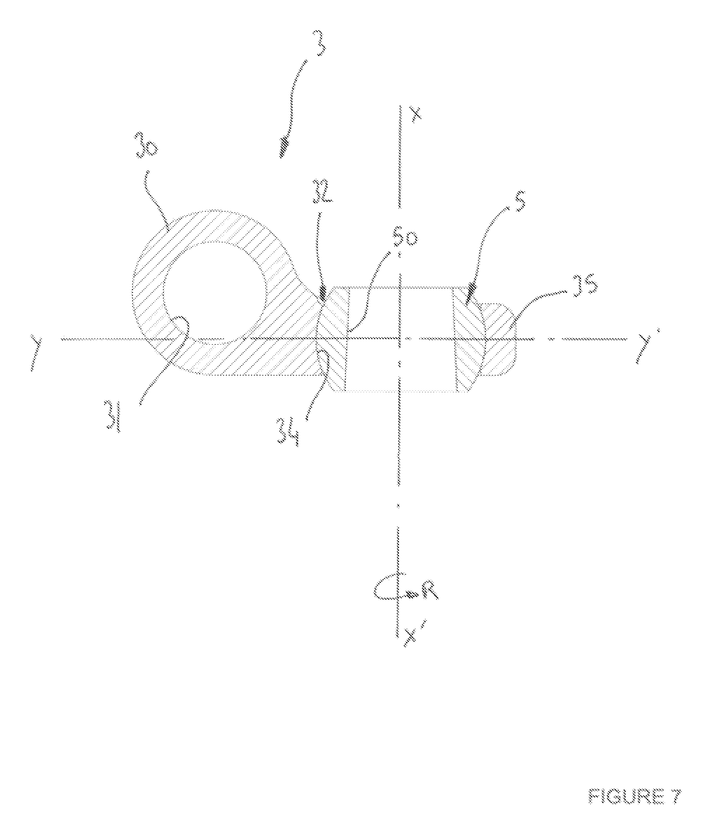

Thus, the sleeve 5 is carried by the main axes xx' and yy' of the second bore 32 and is able to move freely in rotation and in tilting movements inside said second bore (FIGS. 6 and 7).

When the sleeve 5 is arranged in the second bore 32 in a horizontal position parallel to the horizontal plane containing the continuation 35, it is blocked in a vertical direction inside the second bore 32 on account of the complementary outer profiles of said sleeve 5 and of the seat 34.

By contrast, the sleeve 5 is free to move inside the second bore 32 in a rotation movement R and/or in a lateral tilting movement B with respect to the vertical axis xx' of the second bore 32 and/or in a rotation movement R1 with respect to the vertical axis vv' of the anchoring screw 4 when the latter axis is not coincident with that of the second bore 32 (FIGS. 8 and 9).

It will be noted that the sleeve 5, when positioned inside the second bore 32, has a height greater than that of the continuation 35 of the connector 3, in such a way that said sleeve protrudes above and below from said continuation, so as to come into contact with the tightening nut 42 upon immobilization of the anchoring screw 4 in said connector 3.

The anchoring screw 4 is introduced inside the connector 3 in such a way that the second anchoring part 41 passes through the bore 50 of the sleeve 5 until its conical profile 47 engages with said bore 50 of complementary conical profile.

The combination of the two conical profiles allows the anchoring screw 4 to be blocked in the sleeve 5 by a Morse taper and renders them dependent on each other, in order to control the angular movements of the connector 3 about said anchoring screw 4 after its immobilization by means of the nut 42.

Thus, after immobilization of the anchoring screw 4 in the osseous body of the subjacent vertebra V2, the mounting and fixing of the connector 3 around the latter makes it possible, by means of the sleeve 5, to control the positioning of said connector in rotation R and/or in tilting B around the axis xx' as a function of the position of the linking rod 2 and/or of the movements of the vertebrae V1, V2 with respect to each other.

It will be noted that the tightening nut 42 comes to bear on the sleeve 5 arranged inside the second bore 32 of the connector 3, allowing the anchoring screw 4 to be blocked exclusively in a vertical direction inside said sleeve, while ensuring the freedom of movement of said connector 3 with respect to said anchoring screw.

It must also be appreciated that the above description has been given solely by way of example and does not in any way limit the scope of the invention, and replacing the described embodiments by any other equivalent embodiment would not represent a departure from the scope of the invention.

1. Facet arthroplasty device comprising linking rods (2) for linking together connectors (3, 6) that are held respectively in the vertebral bodies of each treated vertebra V by way of anchoring screws (4), characterized in that each connector (3) has means for immobilizing and guiding, inside abore (32), a sleeve (5) that allows said connector (3) freedoms of movement in rotation R, R1 and/or in lateral tilting B about a vertical axis xx' and/or vv' of the anchoring screw (4), which has been fixed beforehand in the corresponding vertebral body.

2. Facet arthroplasty device according to claim 1, characterized in that the sleeve (5) has a spherical outer profile and a continuous inner bore (50) of conical profile.

3. Facet arthroplasty device according to claim 1, characterized in that the linking rod (2) comprises a cylindrical part (20), which is continued by an inclined part (21) having in cross section a square or rectangular profile.

4. Facet arthroplasty device according to claim 3, characterized in that each connector (3) is formed by a body (30) comprising a first bore (31), which allows the cylindrical part (20) of the linking rod (2) to be guided in translation, and a second bore (32), which is formed in a continuation (35) for receiving the sleeve (5) and the anchoring screw (4).

5. Facet arthroplasty device according to claim 4, characterized in that the second bore (32) has two internal vertical indents (33) arranged opposite each other and opening into a seat (34) formed on the inner periphery of the second bore (32).

6. Facet arthroplasty device according to claim 5, characterized in that the indents (33) and the seat (34) formed inside the second bore (32) of each connector (3) have a spherical profile complementing the outer profile of the sleeve (5), in such a way that said sleeve (5) can move freely inside the second bore (32) in a rotation movement R and/or in a lateral tilting movement B with respect to the vertical axis xx' of said bore (32) and/or in a rotation movement R1 with respect to the vertical axis vv' of the anchoring screw (4).

7. Facet arthroplasty device according to claim 4, characterized in that the sleeve (5) has a height greater than that of the continuation (35) of the connector (3).

8. Facet arthroplasty device according to claim 1, characterized in that each anchoring screw (4) has a convex profile (43) between a first anchoring part (40) and a second anchoring part (41), which convex profile (43) is continued, in the direction of the second anchoring part (41), by a conical profile (47) designed to cooperate with the conical bore (50) of the sleeve (5) in order to constitute a Morse taper connection permitting the immobilization of said sleeve (5) on said anchoring screw (4).

9. Facet arthroplasty device according to claim 3, characterized in that the inclined part (21) of the linking rod (2) is held, by way of a locking screw (60), inside connectors (6) that have been fixed beforehand in the superjacent vertebrae V2 of the spinal segment to be treated.

10. Facet arthroplasty device according to claim 1, characterized in that it comprises, between the connectors (3, 6), a transverse linking device (7) composed of connectors (70), which are fixed on the cylindrical part (20) of each linking rod (2) by way of locking screws (71), and of a transverse rod (72) arranged perpendicularly with respect to said linking rod (2) and allowing said connectors (70) to be linked together.
